(19) United States
(12) Patent Application Publication
(10) Pub. No.: US 2010/0049068 A1
(43) Pub. Date: Feb. 25, 2010

(54) IN-VEHICLE ELECTROCARDIOGRAPH DEVICE AND VEHICLE

(76) Inventors: Yoshitaka FUWAMOTO, Toyota-shi (JP); Masaaki Makikawa, Osaka (JP); Yuta Ito, Oumihachiman (JP); Toshiyuki Matsuda, Kusatsu (JP); Takashi Komura, Toyota-shi (JP); Taiji Kawachi, Kariya-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/545,105

(22) Filed: Aug. 21, 2009

(30) Foreign Application Priority Data
Aug. 22, 2008 (JP) 2008-213588

(51) Int. Cl. A61B 5/0408; H01R 43/00

(52) U.S. Cl. 600/509; 29/825

(57) ABSTRACT

An in-vehicle electrocardiograph device includes: a direct electrode that is used to detect an electric potential of a body of a vehicle occupant in a state in which the direct electrode contacts a skin of the occupant; a capacitive-coupled electrode that is used to detect the electric potential of the body of the occupant in a state in which the capacitive-coupled electrode does not contact the skin of the occupant; and an electrocardiographic waveform determination unit that determines an electrocardiographic waveform of the occupant based on an electric potential at the direct electrode and an electric potential at the capacitive-coupled electrode.

### INCORPORATION BY REFERENCE

The disclosure of Japanese Patent Application No. 2008-213588 filed on Aug. 22, 2008 including the specification, drawings and abstract is incorporated herein by reference in its entirety.

### BACKGROUND OF THE INVENTION

1. Field of the Invention

The invention relates to an in-vehicle electrocardiograph device and a vehicle that obtain an electrocardiographic waveform of a vehicle occupant.

2. Description of the Related Art

Electrocardiograms are widely used in various medical settings. An electrocardiogram indicates, in the form of a graph, an electric activity of a heart, which is detected with the use of electrodes for measuring an electric potential of a body.

Researches on the technology for obtaining an electrocardiogram in a vehicle have been conducted. This is because monitoring the activity of the heart of a driver makes it possible to suppress occurrence of various inconveniences due to an abnormality of the driver's heart that is caused while the driver is driving the vehicle.

An example of a device that obtains an electrocardiogram in a vehicle is described in WO 2004/089209. A device according to WO 2004/089209 detects electric potentials of the right hand and the left hand of a driver using a pair of electrodes one of which is formed on a right side-portion of a steering wheel and the other of which is formed on a left side-portion of the steering wheel, and obtains an electrocardiographic waveform based on the difference in electric potential between the right hand and the left hand of the driver.

Japanese Patent Application Publication No. 2007-82938 (JP-A-2007-82938) describes a device that includes a pair of isolated electrodes (capacitive-coupled electrodes) for obtaining an electrocardiographic waveform, and obtains an electrocardiographic waveform based on the difference in electric potential between the isolated electrodes. The isolated electrodes are embedded in a seatbelt in a manner such that the isolated electrodes are opposed to the body of an occupant while being electrically insulated from the occupant when the occupant wears the seatbelt.

The device described in WO 2004/089209 is unable to obtain an electrocardiographic waveform of the driver unless the driver holds the steering wheel with both right and left hands. However, the driver sometimes operates the steering wheel with one hand. Therefore, it is difficult to continuously monitor the driver's heart activity with the use of the device described in WO 2004/089209.

With the device described in JP-A-2007-82938, it is difficult to obtain an accurate electrocardiographic waveform. This is because the distance between the electrodes and the driver's skin fluctuates due to, for example, vibration caused when the vehicle is in motion, and such fluctuations may be a factor for generation of noise in an electrocardiographic waveform.

### SUMMARY OF THE INVENTION

The invention provides an in-vehicle electrocardiograph device and vehicle that is able to obtain an accurate electrocardiographic waveform of a vehicle occupant with less interruption.

A first aspect of the invention relates to an in-vehicle electrocardiograph device which includes: a direct electrode that is used to detect an electric potential of a body of a vehicle occupant in a state in which the direct electrode contacts a skin of the occupant; a capacitive-coupled electrode that is used to detect the electric potential of the body of the occupant in a state in which the capacitive-coupled electrode does not contact the skin of the occupant; and an electrocardiographic waveform determination unit that determines an electrocardiographic waveform of the occupant based on an electric potential at the direct electrode and an electric potential at the capacitive-coupled electrode.

A second aspect of the invention relates to an in-vehicle electrocardiograph device which includes: direct detection means for directly detecting an electric potential a body of a vehicle occupant in a state in which the direct detection means contacts a skin of the occupant; indirect detection means for detecting the electric potential of the body of the occupant in a state in which the indirect detection means does not contact the skin of the occupant; and electrocardiographic waveform determination means for determining an electrocardiographic waveform of the occupant based on an electric potential at the direct detection means and an electric potential at the indirect detection means.

A third aspect of the invention relates to a vehicle which includes: a direct electrode that is used to detect an electric potential of a body of a vehicle occupant in a state in which the direct electrode contacts a skin of the occupant; a capacitive-coupled electrode that is used to detect the electric potential of the body of the occupant in a state in which the capacitive-coupled electrode does not contact the skin of the occupant; and an electrocardiographic waveform determination unit that determines an electrocardiographic waveform of the occupant based on an electric potential at the direct electrode and an electric potential at the capacitive-coupled electrode.

According to the aforementioned aspects of the invention, it is possible obtain a more accurate electrocardiographic waveform of the vehicle occupant with less interruption.

### BRIEF DESCRIPTION OF THE DRAWINGS

The foregoing and further features and advantages of the invention will become apparent from the following description of example embodiments with reference to the accompanying drawings, wherein like numerals are used to represent like elements and wherein:

FIG. 1 is a view showing a configuration example of an in-vehicle electrocardiograph device according to a first embodiment of the invention;

FIG. 2 is a view showing a manner of fitting a direct electrode to a steering wheel;

FIGS. 3A and 3B are views showing examples of a manner of fitting a capacitive-coupled electrode to a vehicle seat;

FIG. 4 is a view showing a specific example of a recording control device together with the direct electrode and the capacitive-coupled electrode;

FIG. 5 is a cross-sectional view showing a configuration of an active electrode;

FIG. 6 is a view showing a virtual circuit configuration according to a second embodiment of the invention;

FIG. 7 is a cross-sectional view showing another example of the configuration of the active electrode;

FIG. 8 is a view showing a configuration example of an in-vehicle electrocardiograph device according to a third embodiment of the invention; and

FIG. 9 is a view showing a configuration example of a recording control device according to the third embodiment of the invention.

### DETAILED DESCRIPTION OF EMBODIMENTS

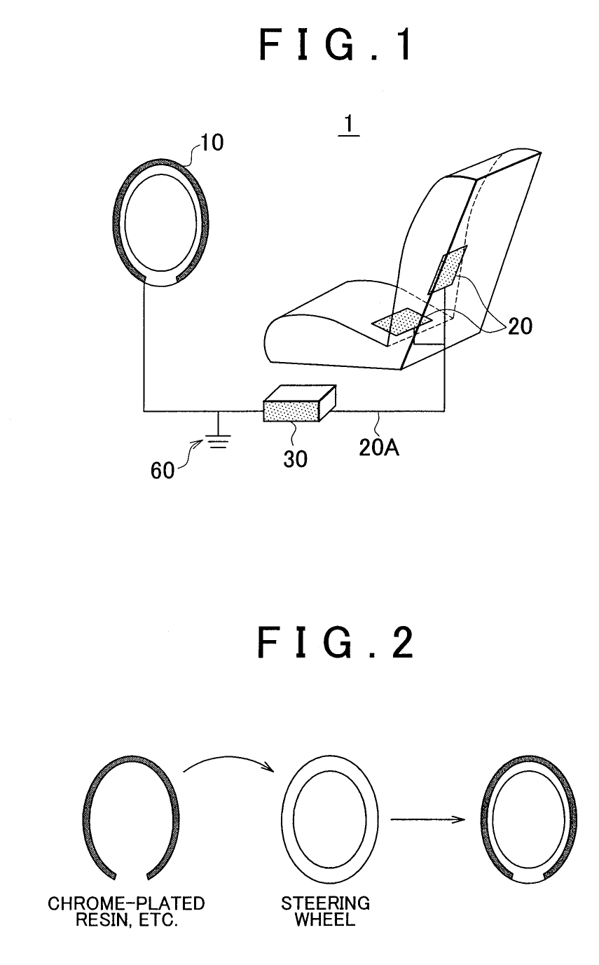

An in-vehicle electrocardiograph device 1 according to a first embodiment of the invention will be described in detail. FIG. 1 is a view showing a configuration example of the in-vehicle electrocardiograph device 1 according to the first embodiment of the invention. The in-vehicle electrocardiograph device 1 includes, as main components, a direct electrode 10, a capacitive-coupled electrode 20, and a recording control device 30. The description below will be provided on the assumption that the in-vehicle electrocardiograph device 1 is a device that obtains an electrocardiographic waveform of a driver of a vehicle. However, the in-vehicle electrocardiograph device 1 according to the first embodiment of the invention may be used to obtain electrocardiographic waveforms of occupants other than the driver (this also applies to second and third embodiments of the invention, which will be described later in detail).

The direct electrode 10 is used to detect an electric potential of the driver's body in the state where the direct electrode 10 contacts the driver's skin. The direct electrode 10 is made of, for example, chrome-plated resin. The direct electrode 10 is fitted to, for example, an outer peripheral face of the steering wheel. FIG. 2 is a view showing a manner of fitting the direct electrode 10 to the steering wheel. The component to which the direct electrode 10 is fitted is not limited to the steering wheel. The direct electrode 10 may be fitted to other components such as a shift lever that the driver directly and frequently touches. The direct electrode 10 is connected to a ground terminal 60.

If the in-vehicle electrocardiograph device 1 is used to obtain an electrocardiographic waveform of an occupant other than the driver, the direct electrode 10 is fitted to, for example, an upper portion of a center console or an operation portion provided on the inner side of a vehicle door.

The capacitive-coupled electrode 20 is used to detect an electric potential of the driver's body through capacitive-coupling in the state where the capacitive-coupled electrode 20 does not contact the driver's skin. The capacitive-coupled electrode 20 is fitted to a vehicle seat. In FIG. 1, the capacitive-coupled electrode 20 includes two electrodes that are disposed so as to face the buttocks and the lower back of the driver, and that are electrically connected to each other. However, the number of the electrodes included in the capacitive-coupled electrode 20 is not limited to two. The capacitive-coupled electrode 20 may include a single electrode, or three or more electrodes that are electrically connected to each other. A signal wire 20A is connected to the capacitive-coupled electrode 20 so that a signal indicating an electric potential of the capacitive-coupled electrode 20, which fluctuates in accordance with fluctuations in the electric potential of the driver's body, is transmitted to the recording control device 30.

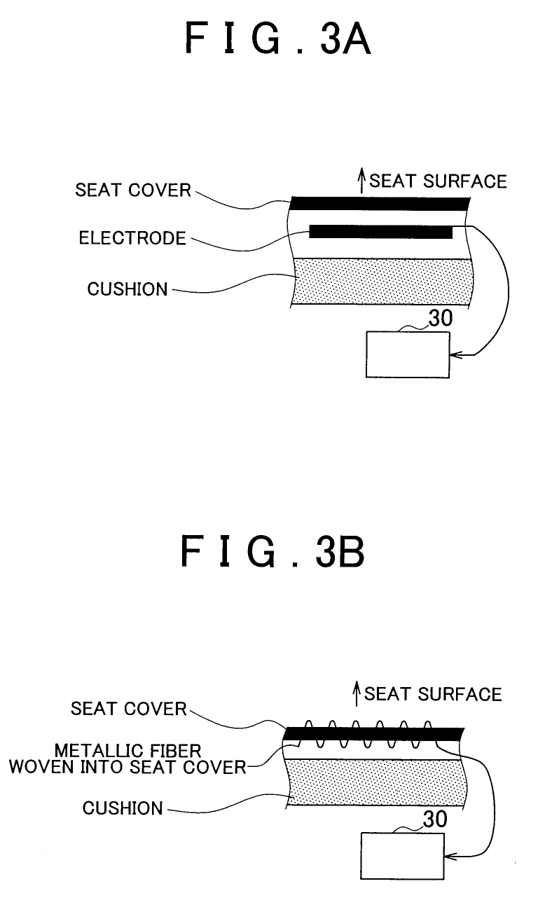

FIG. 3A and FIG. 3B are views showing examples of a manner of fitting the capacitive-coupled electrode 20 to the vehicle seat. The capacitive-coupled electrode 20 may be a plate-shaped electrode that is provided between a seat face and a seat cushion as shown in FIG. 3A. Alternatively, the capacitive-coupled electrode 20 may be a mesh electrode, for example, a metallic fiber woven into the seat face as shown in FIG. 3B.

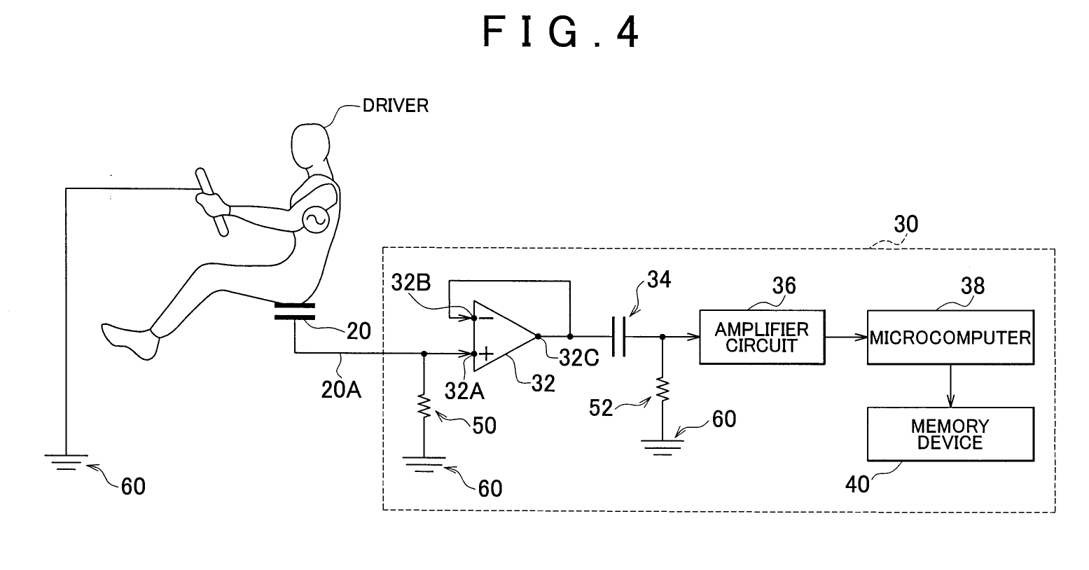

FIG. 4 is a view showing a specific example of the recording control device 30 together with the direct electrode 10 and the capacitive-coupled electrode 20. As shown in FIG. 4, the ground terminal 60 is connected to the signal wire 20A via a resistance 50. This configuration forms a virtual circuit (that functions as a high-pass filter or a band-pass filter) to which a signal indicating the electric potential of the driver's body is input.

The recording control device 30 includes, for example, a voltage follower 32, a capacitor 34, an amplifier circuit 36, a microcomputer 38, and a memory device 40. The configuration may be replaced by, for example, an operational amplifier.

The signal wire 20A is connected to a positive input terminal 32A of the voltage follower 32, and a signal indicating the electric potential of the capacitive-coupled electrode 20 is input in the positive input terminal 32A as a voltage signal. A feedback signal from an output terminal 32C of the voltage follower 32 is input in a negative input terminal 32B of the voltage follower 32. With this configuration, the voltage follower 32 transmits the voltage signal from the capacitive-coupled electrode 20 to the output terminal 32C while limiting electric currents that are input in the voltage follower 32.

The capacitor 34 functions as a high-pass filter or a band-pass filter in cooperation with resistance 52. The amplifier circuit 36 amplifies the voltage signal from which low-frequency components are removed by the capacitor 34 and the resistance 52, and outputs the amplified voltage signal to the microcomputer 38.

The microcomputer 38 samples the voltage signals, received from the amplified circuit 36, at predetermined intervals, and stores the voltage signals in the memory device 40 as time-series data. The memory device 40 is, for example, flash memory. An electrocardiographic waveform is obtained by displaying or printing the time-series data stored in the memory device 40.

This configuration allows the in-vehicle electrocardiograph device 1 to continuously obtain an electrocardiographic waveform of the driver. The driver seldom takes both of his/her hands off the direct electrode 10. In addition, as long as the driver is seated in the driver's seat, it is possible to continuously detect the electric potential of the driver's body with the use of the capacitive-coupled electrode 20 fitted to the vehicle seat. Therefore, it is possible to obtain an electrocardiographic waveform of the driver with less interruption when the electrocardiograph device 1 according to the first embodiment of the invention is used than when the device described in, for example, WO 2004/089209 is used. The device described in WO 2004/089209 includes a pair of electrodes one of which is formed on the right side-portion of the steering wheel and the other of which is formed on the left side-portion of the steering wheel, and obtains electric potentials of the right hand and the left hand of the driver. Therefore, if the driver holds the steering wheel with only one hand, the electrocardiographic waveform is interrupted.

Further, the in-vehicle electrocardiograph device 1 obtains a more accurate electrocardiographic waveform of the driver. If an electrocardiographic waveform is obtained based on the difference in electric potential between a pair of capacitive-coupled electrodes with the use of, for example, the device described in JP-A-2007-82938, the distance between the driver's body and the capacitive-coupled electrodes fluctuates due to, for example, vibration caused when the vehicle is in motion, and such fluctuations may be a factor for generation of noise. Therefore, it is difficult to obtain an accurate electrocardiographic waveform. In contrast, according to the first embodiment of the invention, the electric potential of the direct electrode 10 is used as one of the reference electric potentials used to calculate the electric potential difference. Therefore, the influence of the fluctuations in the distance between the driver's body and the capacitive-coupled electrode is reduced. Further, according to the first embodiment of the invention, because the capacitive-coupled electrode 20 is fitted to the vehicle seat instead of a component that moves greatly, for example, a seatbelt, fluctuations in the distance between the driver's body and the capacitive-coupled electrode 20 is suppressed. Accordingly, it is possible to obtain a more accurate electrocardiographic waveform of the driver.

With the in-vehicle electrocardiograph device 1 according to the first embodiment of the invention described above, it is possible to obtain a more accurate electrocardiographic waveform of the vehicle occupant with less interruption.

Next, an in-vehicle electrocardiograph device according to a second embodiment of the invention will be described. The in-vehicle electrocardiograph device according to the second embodiment of the invention includes an active electrode 70 in which a capacitive-coupled electrode and an amplifier are integrally formed. Other components, that is, the direct electrode 10, the microcomputer 38, and the memory device 40 may be the same as those in the first embodiment of the invention.

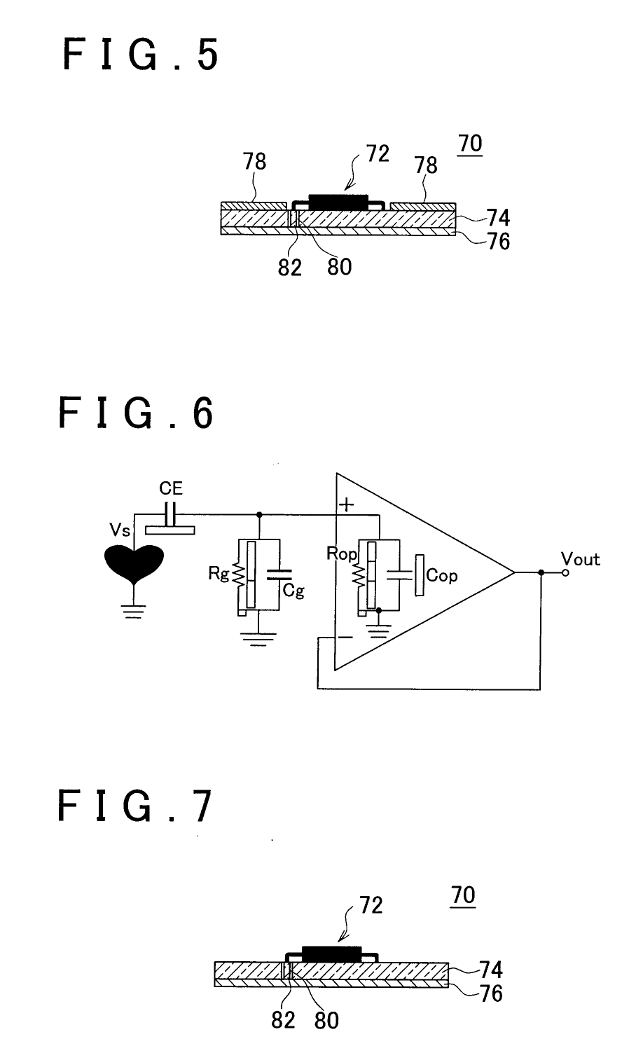

FIG. 5 is a cross-sectional view showing the configuration of the active electrode 70. In the active electrode 70, a preamplifier 72 is fitted on a glass substrate 74, a capacitive-coupled electrode 76 is fitted to the glass substrate 74 on a face opposite to the face on which the preamplifier 72 is fitted, and shield electrodes 78 are fitted to the glass substrate 74 on the face on which the preamplifier 72 is fitted. The shield electrodes 78 are used to eliminate the influence of an electric field on the preamplifier 72 and to maintain physical strength of the glass substrate 74. The capacitive-coupled electrode 76 is connected to the preamplifier 72 via a leading wire 82 fitted in a through-hole 80.

FIG. 6 shows a virtual circuit configuration formed of the configuration described above. “Vs” in FIG. 6 represents an electric potential difference in the driver's body (more specifically, the electric potential difference between the driver's hand(s) and the driver's buttocks or lower back). “CE” in FIG. 6 represents a capacity of a virtual capacitor formed by the driver's body and the capacitive-coupled electrode 76. “Rg” in FIG. 6 represents a resistance for discharging static electricity produced due to movement of the driver's body. “Cg” in FIG. 6 represents a capacity of a virtual capacitor formed by the capacitive-coupled electrode 76 and the shield electrodes 78. “Rop” and “Cop” in FIG. 6 represent an input resistance and an input capacitance of the preamplifier 72, respectively.

A gain G (S) of the virtual circuit configuration is expressed by Equation 1 indicated below. As indicated by Equation 1, the circuit configuration functions as a high-pass filter, and the cutoff frequency thereof is determined based on four parameters CE, Cg, Cop, and Rg.

$$G ( s ) = \frac{CE \cdot Rg \cdot S}{1 + ( CE + Cg + Cop ) \cdot Rg \cdot S} \qquad ( 1 )$$

Equation 1 may be applied to the first embodiment of the invention.

With the in-vehicle electrocardiograph device according to the second embodiment of the invention, a more accurate electrocardiographic waveform of the vehicle driver is obtained with less interruption, as in the first embodiment of the invention. Further, in the in-vehicle electrocardiograph device according to the second embodiment of the invention, because the preamplifier 72 and the capacitive-coupled electrode 76 are fitted to the same substrate, it is possible to make hardware of the in-vehicle electrocardiograph device more compact.

The active electrode 70 may be configured without the shield electrodes 78 as shown in FIG. 7.

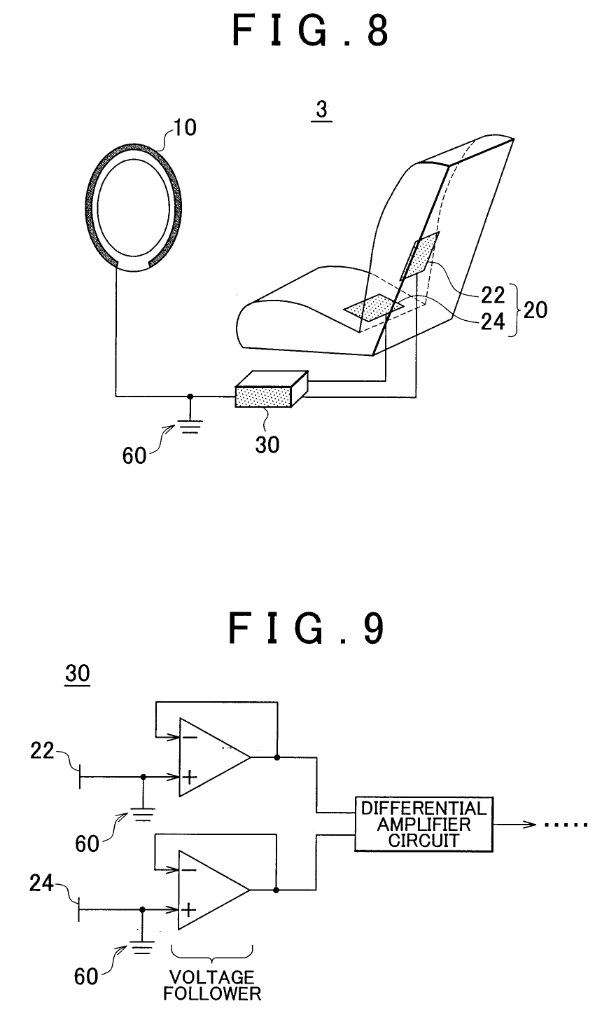

Next, an in-vehicle electrocardiograph device 3 according to a third embodiment of the invention will be described. FIG. 8 is a configuration example of the in-vehicle electrocardiograph device 3 according to the third embodiment of the invention. The in-vehicle electrocardiograph device 3 includes, as main components, the direct electrode 10, the capacitive-coupled electrode 20, and the recording control device 30.

The description of the direct electrode 10 will not be provided below because the direct electrode 10 is the same as that in the first embodiment of the invention.

The capacitive-coupled electrode 20 includes a plurality of electrodes, that is, electrodes 22 and 24, and signals indicating electric potentials of the electrodes 22 and 24 are output to the recording control device 30.

FIG. 9 is a configuration example of the recording control device 30 according to the third embodiment of the invention. The recording control device 30 according to the third embodiment of the invention includes two voltage followers one of which is connected to the electrode 22 and the other of which is connected to the electrode 24, and a differential amplifier circuit that outputs the difference between the outputs from the two voltage followers. The two voltage followers each use the ground potential, that is, the electric potential of the direct electrode 10, as the reference electric potential.

With this configuration, because the reference electric potential does not fluctuate, it is possible to more accurately detect the difference in electric potential between the electrodes 22 and 24. Further, an electrocardiographic waveform of the driver is obtained with less interruption, as in the first embodiment of the invention.

With the in-vehicle electrocardiograph device 3 according to the third embodiment of the invention, it is possible to obtain a more accurate electrocardiographic waveform of the vehicle driver with less interruption.

The electrocardiographic waveform thus obtained is displayed or printed so that the driver is able to check his/her electrocardiographic waveform. Further, whether an abnormality is present in the electrocardiographic waveform may be determined with the use of, for example, the microcomputer 38. If it is determined that there is an abnormality, drive assist control, for example, control for gradually placing the vehicle into a halt is executed. Still further, data on the electrocardiographic waveform may be sent to a facility outside of the vehicle via wireless radio communication, and whether an abnormality is present in the electrocardiographic waveform may be determined at the outside facility.

Next, a specific example of a method of monitoring an electrocardiographic waveform with the use of, for example, the microcomputer 38 will be described. The microcomputer 38 determines whether the driver's heartbeat is arrhythmic based on the time-series data stored in the memory device 40.

The recording control device 30 includes a communication wire so that outputs from a vehicle speed sensor, etc., are input in the recording control device 30. If the microcomputer 38 determines that the vehicle is in motion based on the output from for example, the vehicle speed sensor, the driver's heart condition is roughly determined based on, for example, interval between peaks of a R-wave (wave height of the R wave) contained in the electrocardiographic waveform. On the other hand, if the microcomputer 38 determines that the vehicle is in a halt, the driver's heart condition is more precisely determined based on the entire electrocardiographic waveform including the peaks of the R wave.

The electrocardiographic waveform is formed mainly of a P wave that reflects electrical excitation of atriums of the heart, a Q wave, an R wave, and an S wave (hereinafter collectively referred to as “QRS complex”) that reflect electrical excitation of ventricles of the heart, and a T wave that reflects a process of repolarization of the cardiomyocyte of the excited ventricles. The wave height (potential difference) of the R wave is the greatest, and therefore, the R wave has the highest resistance to noise, for example, myoelectric potential. The wave height of the T wave is the second greatest, and the wave height of the P wave is the smallest.

Therefore, when the vehicle is in motion, the driver's heart condition is roughly determined based on, for example, the interval between the peaks of the R wave that has the greatest wave height in the electrocardiographic waveform. On the other hand, when the vehicle is in a halt, the driver's heart condition is more precisely determined based on the entire electrocardiographic waveform that includes the T wave and P wave that have smaller wave heights than that of the R wave. This is because the driver is more easily placed in the resting state, and therefore, noise, for example, myoelectric potential is reduced when the vehicle is in a halt.

The microcomputer 38 determines whether the driver's heartbeat is arrhythmic in a method that is suitable for the vehicle condition as described above.

When the vehicle is in motion, the microcomputer 38 detects only the peaks of the R wave from the electrocardiographic waveform, and calculates the heart rate per minute based on the interval between the peaks. Then, the microcomputer 38 monitors fluctuations in the heart rate based on the R-R interval (RRI), and conducts frequency-analysis of the fluctuations in the heart rate to calculate a low frequency component (LF) and a high frequency component (HF). Then, the microcomputer 38 determines based on the calculated heart rate and the LF/HF ratio whether the driver's heartbeat is arrhythmic (there is a possibility that the driver's heartbeat is arrhythmic). More specifically, the microcomputer 38 determines that there is a possibility that the driver's heartbeat is arrhythmic if at least one of the following conditions is satisfied: the condition that the heart rate increases by 25% or more from the average value of the heart rate within preceding 5 minutes; the condition that the heart rate is equal to or higher than 100 beats per minute or equal to or lower than 40 beats per minute; and the condition that the LF/HF ratio increases by 50% or more from the LF/HF ratio within a preceding period of 30 minutes to 40 minutes. If it is determined that there is a possibility that the driver's heartbeat is arrhythmic, the microcomputer 38 executes interference control on a drive unit and a brake system of the vehicle so as to execute control for gradually bringing the vehicle to a halt or control for displaying a warning on a display screen.

When the vehicle is in a halt, the microcomputer 38 detects the wave height of a ST wave from the electrocardiographic waveform, and estimates the blood pressure based on a pulse waveform. Then, the microcomputer 38 determines whether the driver's heartbeat is arrhythmic (whether there is a possibility that the driver's heartbeat is arrhythmic) based on the detected wave height of the ST wave or the estimated blood pressure. More specifically, the microcomputer 38 determines that the driver's heartbeat is arrhythmic (there is a possibility that the driver's heartbeat is arrhythmic) if at least one of the following conditions is satisfied: the condition that the wave height of the ST wave is equal to or greater than a value obtained by adding 0.02 mV to the reference electric potential (+0.02 mV) or equal to or lower than a value obtained by subtracting 0.02 mV from the reference electric potential (−0.02 mV); or the condition that the blood pressure increases or decreases by 25% or more. If it is determined that there is a possibility that that the driver's heartbeat is arrhythmic, the microcomputer 38 automatically reports the possibility of arrhythmia to a pre-registered contact address, for example, a member of the family, a family doctor, or help network via a communication device. Alternatively, the microcomputer 38 sounds a horn or blinks light so as to alert people outside of the vehicle to an emergency.

A template that contains a reference electrocardiographic waveform may be stored in a memory device (not shown), such as a random access memory (RAM) or a hard disk drive (HDD), and, for example, whether there is a possibility that the driver's heartbeat is arrhythmic may be determined by comparing the obtained electrocardiographic waveform and the reference electrocardiographic waveform contained in the template. In this case, it is possible to determine whether atrial fibrillation, sinus arrhythmia, or premature atrial contraction has occurred. The template may be updated, or deleted, or a new template may be registered based on the electrocardiographic waveform obtained by the in-vehicle electrocardiograph device.

While the invention has been described with reference to example embodiments thereof, it is to be understood that the invention is not limited to the described embodiments or constructions. To the contrary, the invention is intended to cover various modifications and equivalent arrangements. In addition, while the various elements of the example embodiments are shown in various combinations and configurations, other combinations and configurations, including more, less or only a single element, are also within the scope of the invention.

What is claimed is:

1. An in-vehicle electrocardiograph device, comprising:
a direct electrode that is used to detect an electric potential of a body of a vehicle occupant in a state in which the direct electrode contacts a skin of the occupant;
a capacitive-coupled electrode that is used to detect the electric potential of the body of the occupant in a state in which the capacitive-coupled electrode does not contact the skin of the occupant; and
an electrocardiographic waveform determination unit that determines an electrocardiographic waveform of the occupant based on an electric potential at the direct electrode and an electric potential at the capacitive-coupled electrode.

2. The in-vehicle electrocardiograph device according to claim 1, wherein the electrocardiographic waveform determination unit determines the electrocardiographic waveform of the occupant based on a difference between the electric potential at the direct electrode and the electric potential at the capacitive-coupled electrode.

3. The in-vehicle electrocardiograph device according to claim 1, wherein the direct electrode includes one electrode.

4. The in-vehicle electrocardiograph device according to claim 1, wherein:
the electrocardiographic waveform determination unit includes an amplifier that amplifies a signal indicating the electric potential at the capacitive-coupled electrode; and
the electrocardiographic waveform determination unit determines the electrocardiographic waveform of the occupant based on the amplified signal indicating the electric potential at the capacitive-coupled electrode.

5. The in-vehicle electrocardiograph device according to claim 4, wherein the capacitive-coupled electrode is integrally formed with the amplifier.

6. The in-vehicle electrocardiograph device according to claim 1, wherein:
the direct electrode is connected to a ground;
the capacitive-coupled electrode includes a plurality of electrodes; and
the electrocardiographic waveform determination unit uses the electric potential at the direct electrode as a reference electric potential, and determines the electrocardiographic waveform of the occupant based on a difference in electric potential between the plurality of electrodes included in the capacitive-coupled electrode.

7. The in-vehicle electrocardiograph device according to claim 6, wherein each one of the plurality of electrodes is connected to the ground.

8. The in-vehicle electrocardiograph device according to claim 1, wherein:
the direct electrode is directly connected to a ground;
the capacitive-coupled electrode is connected to the ground via a resistance; and
the electrocardiographic waveform determination unit uses the electric potential at the direct electrode as a reference electric potential, and determines the electrocardiographic waveform of the occupant based on a difference between the reference electric potential and the electric potential at the capacitive-coupled electrode.

9. The in-vehicle electrocardiograph device according to claim 1, wherein the direct electrode is fitted to at least a steering wheel.

10. The in-vehicle electrocardiograph device according to claim 1, wherein the direct electrode is fitted to at least a shift lever.

11. The in-vehicle electrocardiograph device according to claim 1, wherein the direct electrode is fitted to at least an upper portion of a center console.

12. The in-vehicle electrocardiograph device according to claim 1, wherein the direct electrode is fitted to at least an interior member of the vehicle.

13. The in-vehicle electrocardiograph device according to claim 1, wherein the capacitive-coupled electrode is provided in at least an inner portion of a vehicle seat.

14. The in-vehicle electrocardiograph device according to claim 1, wherein the capacitive-coupled electrode is fitted to a substrate on a face opposite to a face on which an amplifier, which amplifies a signal indicating the electric potential of the body of the occupant detected with use of the capacitive-coupled electrode, is fitted, and the capacitive-coupled electrode is connected to the amplifier via a through-hole formed in the substrate.

15. The in-vehicle electrocardiograph device according to claim 14, wherein a shield electrode, which eliminates an influence of an electric field on the amplifier and which strengthens the substrate, is fitted to the substrate on the face on which the amplifier is fitted.

16. The in-vehicle electrocardiograph device according to claim 1, further comprising:
a determination unit that determines whether heartbeat of the occupant is arrhythmic or whether there is a possibility that the heartbeat of the occupant is arrhythmic based on the electrocardiographic waveform of the occupant determined by the electrocardiographic waveform determination unit.

17. The in-vehicle electrocardiograph device according to claim 16, further comprising:
a vehicle speed detection unit that detects a vehicle speed,
wherein the determination performs determination in different manners depending on whether the vehicle is in motion or the vehicle is in a halt, which is determined based on the vehicle speed detected by the vehicle speed detection unit.

18. The in-vehicle electrocardiograph device according to claim 17, wherein:
when the vehicle is in motion, the determination unit determines whether the heartbeat of the occupant is arrhythmic or whether there is a possibility that the heartbeat of the occupant is arrhythmic by monitoring a fluctuation in a heart rate of the occupant; and
when the vehicle is in a halt, the determination unit determines whether the heartbeat of the occupant is arrhythmic or whether there is a possibility that the heartbeat of the occupant is arrhythmic based on the electrocardiographic waveform.

19. An in-vehicle electrocardiograph device, comprising:
direct detection means for directly detecting an electric potential a body of a vehicle occupant in a state in which the direct detection means contacts a skin of the occupant;
indirect detection means for detecting the electric potential of the body of the occupant in a state in which the indirect detection means does not contact the skin of the occupant; and
electrocardiographic waveform determination means for determining an electrocardiographic waveform of the occupant based on an electric potential at the direct detection means and an electric potential at the indirect detection means.

20. A vehicle comprising:
a direct electrode that is used to detect an electric potential of a body of a vehicle occupant in a state in which the direct electrode contacts a skin of the occupant;
a capacitive-coupled electrode that is used to detect the electric potential of the body of the occupant in a state in which the capacitive-coupled electrode does not contact the skin of the occupant; and
an electrocardiographic waveform determination unit that determines an electrocardiographic waveform of the occupant based on an electric potential at the direct electrode and an electric potential at the capacitive-coupled electrode.
